(19) United States
(12) Patent Application Publication
(10) Pub. No.: US 2021/0137491 A1
(43) Pub. Date: May 13, 2021

(54) INFORMATION SELECTION DEVICE

(71) Applicants: TERUMO KABUSHIKI KAISHA, Shibuya-ku, Tokyo (JP); Rokken Inc., Sakai-shi, Osaka (JP)

(72) Inventors: Yasukazu SAKAMOTO, Hiratsuka-shi, Kanagawa (JP); Katsuhiko SHIMIZU, Fujinomiya-shi, Shizuoka (JP); Ryosuke SAGA, Osaka-shi, Osaka (JP)

(73) Assignees: TERUMO KABUSHIKI KAISHA, Shibuya-ku, Tokyo (JP); Rokken Inc., Sakai-shi, Osaka (JP)

(21) Appl. No.: 17/043,147

(22) PCT Filed: Mar. 27, 2019
(86) PCT No.: PCT/JP2019/013474

§ 371 Date: Sep. 29, 2020

(30) Foreign Application Priority Data
Mar. 29, 2018 (JP) 2018-066171

(51) Int. Cl. A61B 8/12; A61B 8/00; A61B 8/02

(52) U.S. Cl. CPC A61B 8/12; A61B 8/466; A61B 8/02; A61B 8/467

(57) ABSTRACT

An information selection device includes a pulsation input unit capable of inputting a pulsation cycle of a heart of a subject, a respiration input unit capable of inputting a respiration cycle of the subject, an information input unit capable of inputting inner wall surface information on the heart at different times, and a control unit that selects, as selection information, the inner wall surface information input at a timing at which a predetermined pulsation phase range included in the pulsation cycle and a predetermined respiration phase range included in the respiration cycle overlap each other, out of a plurality of the input inner wall surface information.

### TECHNICAL FIELD

The present disclosure relates to an information selection device.

### BACKGROUND ART

In the related art, a medical instrument is inserted into a heart to perform a treatment on the heart. For example, PTL 1 discloses a technique for generating a three-dimensional image of the heart or a blood vessel and the like. An internal state of the heart can be recognized by using the three-dimensional image of the heart which is generated in this way.

### CITATION LIST

### Patent Literature

[PTL 1] International Publication No. 2016/140116

### SUMMARY OF INVENTION

### Technical Problem

Incidentally, a size or a shape of a heart is changed in a time-dependent manner due to influence of pulsation and the like. Therefore, when mapping is performed on the heart, based on information acquired in the time-dependent manner, it is necessary to consider a time-dependent changes in the size or the shape of the heart. According to a technique disclosed in PTL 1, it is possible to generate a three-dimensional image in which the influence of the pulsation of the heart is reduced. However, there is still room for improvement in performing the mapping on the heart, which considers the time-dependent change in the size or the shape of the heart.

In view of the above-described problem, an object of the present disclosure is to provide an information selection device capable of improving accuracy in performing mapping on a heart, which considers a time-dependent change in a size or a shape of the heart.

### Solution to Problem

According to a first aspect of the present invention, there is provided an information selection device including a pulsation input unit capable of inputting a pulsation cycle of a heart of a subject, a respiration input unit capable of inputting a respiration cycle of the subject, an information input unit capable of inputting inner wall surface information on the heart at different times, and a control unit that selects, as selection information, the inner wall surface information input at a timing at which a predetermined pulsation phase range included in the pulsation cycle and a predetermined respiration phase range included in the respiration cycle overlap each other, out of a plurality of the input inner wall surface information.

In the information selection device according to an embodiment of the present invention, the predetermined pulsation phase range may include a peak of the pulsation cycle.

In the information selection device according to an embodiment of the present invention, the predetermined respiration phase range may include a peak of the respiration cycle.

In the information selection device according to an embodiment of the present invention, the control unit may generate a three-dimensional image of the heart, based on a plurality of the selection information.

In the information selection device according to an embodiment of the present invention, the control unit may classify the inner wall surface information input at a timing at which each of a plurality of pulsation phase ranges having one predetermined pulsation phase range and each of a plurality of respiration phase ranges having one predetermined respiration phase range overlap each other, out of the plurality of inner wall surface information, into a group for each combination.

In the information selection device according to an embodiment of the present invention, the control unit may generate a three-dimensional image of the heart, based on a plurality of inner wall surface information classified into the same group.

The information selection device according to an embodiment of the present invention may further include a display unit. Each time the inner wall surface information is input to the information input unit, the control unit may generate the three-dimensional image of the heart, based on the plurality of inner wall surface information belonging to a group into which the inner wall surface information is classified, and displays the three-dimensional image on the display unit.

The information selection device according to an embodiment of the present invention may further include a display unit. Each time the inner wall surface information belonging to a pre-selection group is input to the information input unit, the control unit may generate the three-dimensional image of the heart, based on a plurality of information belonging to the pre-selection group, and displays the three-dimensional image on the display unit.

According to a second aspect of the present invention, there is provided an information selection device including a pulsation input unit capable of inputting a pulsation cycle of a heart of a subject, a respiration input unit capable of inputting a respiration cycle of the subject, and an information input unit capable of inputting inner wall surface information on the heart at different times. The information input unit receives an input of only the inner wall surface information at a timing at which a predetermined pulsation phase range included in the pulsation cycle and a predetermined respiration phase range included in the respiration cycle overlap each other.

In the information selection device according to an embodiment of the present invention, the information input unit may receive an input of the inner wall surface information acquired by an ultrasound element or an image sensor element located inside a tubular member inserted into the heart. The information selection device may further include a drive unit that moves the ultrasound element or the image sensor element inside the tubular member.

In the information selection device according to an embodiment of the present invention, the information input unit may include the ultrasound element. The ultrasound element may irradiate an inner wall surface of the heart with an ultrasound wave, and may be capable of acquiring the inner wall surface information on the heart, based on the ultrasound wave reflected from the inner wall surface of the heart.

In the information selection device according to an embodiment of the present invention, the drive unit may be connected with a proximal portion of a shaft. The ultrasound element may be fixed to a distal portion of the shaft. The drive unit may move the ultrasound element via the shaft.

### Advantageous Effects of Invention

According to the information selection device of the present disclosure, it is possible to improve accuracy in performing mapping on a heart, which considers a time-dependent change in a size or a shape of the heart.

### BRIEF DESCRIPTION OF DRAWINGS

FIG. 1 is a block diagram illustrating a schematic configuration of an image processing device according to an embodiment of the present invention.

FIG. 2 is a schematic view of the image processing device illustrated in FIG. 1.

FIG. 3 is a perspective view illustrating an example of a medical device into which an ultrasound element is inserted.

FIG. 4 is a sectional view of the medical device illustrated in FIG. 3.

FIG. 5 is a flowchart illustrating details of a method for generating a three-dimensional image of a heart.

FIG. 6(a) is a graph illustrating a time change in intensity of an arterial pulse, and FIG. 6(b) is a graph illustrating a time change in intensity of an electric potential which results from electrical activity of the heart.

FIG. 7 is a graph obtained by compressing the graph illustrated in FIG. 6(a) or 6(b) along a time axis.

FIG. 8 is a view illustrating a distribution of inner wall surface information on the heart which is acquired by using an ultrasound element.

FIG. 9 is a view illustrating a state of selecting selection information from the inner wall surface information on the heart.

FIG. 10 is a view illustrating a distribution of the selection information.

FIG. 11 is a view illustrating a distribution of the inner wall surface information on the heart on which a complementary process is performed.

FIG. 12 is a flowchart illustrating a modification example of a method for generating a three-dimensional image of the heart.

FIG. 13 is a view for describing a pulsation phase range.

FIG. 14 is a view for describing a respiration phase range.

FIG. 15 is a view illustrating a timing at which a predetermined pulsation phase range included in a pulsation cycle and a predetermined respiration phase range included in a respiration cycle overlap each other.

### DESCRIPTION OF EMBODIMENTS

Hereinafter, an embodiment according to the present invention will be described with reference to the drawings. In each drawing, the same reference numerals will be assigned to common configuration elements. In addition, in the present specification, a side of a medical device 2 to be inserted into a biological lumen will be referred to as a “distally located side” or a “distal side”, and an operating hand-side will be referred to as a “proximally located side” or a “proximal side”. In the present embodiment, an image processing device 1 will be described as an embodiment of an information selection device according to the present invention.

### [Image Processing Device 1]

FIG. 1 is a block diagram illustrating a schematic configuration of the image processing device 1 according to an embodiment of the present invention. FIG. 2 is a schematic view of the image processing device 1. As illustrated in FIGS. 1 and 2, the image processing device 1 includes a drive unit 50, a display unit 51, an operation reception unit 52, a storage unit 53, a control unit 54, a pulsation input unit 60, a respiration input unit 70, and an information input unit 76.

As illustrated in FIG. 1, the pulsation input unit 60 is electrically connected to an external pulsation measurement device 160. The respiration input unit 70 is electrically connected to an external respiration measurement device 170. The information input unit 76 is electrically connected to an ultrasound element 21 of an external ultrasound tester 20. The ultrasound tester 20 includes the ultrasound element 21, a shaft 22, and a tube 23.

The drive unit 50 has a built-in motor, and causes the ultrasound element 21 as a peripheral information acquisition device to reciprocate along the extending direction of a catheter 40 (to be described later) via the shaft 22 of the ultrasound tester 20. Specifically, as illustrated in FIG. 2, the drive unit 50 fixes the ultrasound tester 20, and mounts the ultrasound tester 20 on a base 59. The drive unit 50 can reciprocate along the extending direction of the ultrasound tester 20 (that is, the extending direction of the catheter 40 to be described later) with respect to the base 59. Therefore, since the drive unit 50 itself reciprocates along the extending direction of the ultrasound tester 20, the drive unit 50 can cause the ultrasound element 21 to reciprocate along the extending direction of the catheter 40. Furthermore, the drive unit 50 may rotate the ultrasound element 21 in the circumferential direction of the catheter 40 while causing the ultrasound element 21 to reciprocate. In this case, the drive unit 50 may continuously rotate the ultrasound element 21 in one direction, or may cause the ultrasound element 21 to oscillate while repeatedly changing a rotation direction.

The display unit 51 displays and outputs display information generated by the control unit 54. The display unit 51 includes a display device such as a liquid crystal display or an organic EL display, for example.

The operation reception unit 52 receives an input of information or an instruction from an operator, and outputs the received input information or input instruction to the control unit 54. The operation reception unit 52 includes an input device such as a keyboard, a mouse, or a touch panel, for example. In a case where the operation reception unit 52 includes the touch panel, the touch panel may be disposed integrally with the display unit 51.

The storage unit 53 stores various information and programs for causing the control unit 54 to execute a specific function. In addition, the storage unit 53 stores the three-dimensional image of a heart of a subject which is generated by the control unit 54. The storage unit 53 includes a storage device such as a RAM or a ROM, for example.

The control unit 54 controls an operation of each configuration element that configures the image processing device 1. The control unit 54 executes a specific function by reading a specific program. The control unit 54 includes a processor, for example.

The pulsation input unit 60 receives an input of a pulsation cycle of the heart of the subject which is measured by the pulsation measurement device 160. In other words, the pulsation measurement device 160 measures the pulsation cycle of the heart of the subject, and transmits information on the measured pulsation cycle to the pulsation input unit 60. For example, the pulsation measurement device 160 is configured to include an electrocardiograph that acquires an electrocardiogram waveform of the subject, or an arterial pulse measurement device that acquires an arterial pulse of the subject.

The respiration input unit 70 receives an input of a respiration cycle of the subject which is measured by the respiration measurement device 170. In other words, the respiration measurement device 170 measures the respiration cycle of the subject, and transmits information on the measured respiration cycle to the respiration input unit 70. For example, the respiration measurement device 170 is configured to include an electrocardiograph or an arterial pulse measurement device which is common to that of the pulsation measurement device 160. A method for measuring the respiration cycle in a case where the respiration measurement device 170 is configured to include the electrocardiograph or the arterial pulse measurement device which is common to that of the pulsation measurement device 160 will be described later.

The information input unit 76 receives an input of information on an inner wall surface of the heart (hereinafter, simply referred to as “inner wall surface information”) from the ultrasound element 21 as the peripheral information acquisition device. Specifically, the information input unit 76 is electrically connected to the ultrasound element 21 via a signal line extending inside the shaft 22, acquires a signal relating to the inner wall surface information acquired by the ultrasound element 21, and transmits the signal to the control unit 54.

For example, the ultrasound element 21 is located in a distal end of the ultrasound tester 20, and transmits and receives ultrasound wave. In a case where the ultrasound element 21 is located inside the heart, the ultrasound element 21 irradiates the inner wall surface of the heart with the ultrasound wave, and can acquire the inner wall surface information, based on the ultrasound wave reflected from the inner wall surface. The shaft 22 is a flexible linear member which fixes the ultrasound element 21 in the distal portion and is connected with the drive unit 50 in the proximal end. The tube 23 is a flexible tubular member that covers the shaft 22 in the circumferential direction. For example, the ultrasound element 21 is used by being inserted into the catheter 40 of the medical device 2 (to be described later). The ultrasound tester 20 and the medical device 2 (to be described later) may collectively configure one ultrasound catheter.

### [Medical Device 2]

FIG. 3 is a perspective view illustrating an example of the medical device 2 in which the ultrasound element 21 is inserted. FIG. 4 is a sectional view of the medical device 2.

As illustrated in FIGS. 3 and 4, the medical device 2 includes a guide wire 10, electrodes 30a to 30j, and the catheter 40 as a tubular member. The catheter 40 internally defines a first lumen 41 into which the ultrasound tester 20 can be interpolated, and a second lumen 42 into which the guide wire 10 can be interpolated. FIGS. 3 to 5 illustrate a state where the ultrasound tester 20 is interpolated into the first lumen 41 and the guide wire 10 is interpolated into the second lumen 42. Hereinafter, unless otherwise specified, an example will be described in which the ultrasound tester 20 and the guide wire 10 are in states of being respectively interpolated into the first lumen 41 and the second lumen 42. The medical device 2 may not necessarily include the guide wire 10.

The guide wire 10 extends from the proximally located side to the distally located side. The guide wire 10 has a linear portion 11 and an annular expansive portion 12 disposed in an end portion on the distally located side connected with the linear portion 11. For example, the annular expansive portion 12 is made of metal such as a shape memory alloy, and is shape-memorized to annularly expand under an environment in which an external force acts in a prescribed or lower level.

In the state illustrated in FIG. 3, the annular expansive portion 12 is located on the distally located side from the distal end of the second lumen 42, and expands in an annular shape. The annular expansive portion 12 expands outward in a radial direction A of the linear portion 11 of the guide wire 10, and extends along a circumferential direction B of the linear portion 11 of the guide wire 10. The annular expansive portion 12 has an outer diameter larger than that of the catheter 40 in an annularly expanded state. In addition, as illustrated in FIG. 3, in a case of being viewed from the distal side of the guide wire 10, the linear portion 11 of the guide wire 10 and the catheter 40 are located inside an annular shape of the annular expansive portion 12 that expands to have an annular shape larger than the outer diameter of the catheter 40. Hereinafter, even in a state where the annular expansive portion 12 annularly expands, the radial direction of the linear portion 11 of the guide wire 10 will be simply referred to as the “radial direction A”, and the circumferential direction of the linear portion 11 of the guide wire 10 will be simply referred to as the “circumferential direction B”. In addition, hereinafter, unless otherwise specified, description will be made on an assumption that the annular expansive portion 12 is in an annularly expanded state.

The electrodes 30a to 30j are fixed to the annular expansive portion 12, and are fixed at different positions in the extending direction of the annular expansive portion 12, that is, along the circumferential direction B of the guide wire 10. Hereinafter, in a case where the electrodes 30a to 30j are not distinguished from each other, all of these will be collectively referred to as a electrode 30.

The electrode 30 can detect electrical characteristics of an inner wall of a biological lumen by coming into contact with the inner wall of the biological lumen. For example, as the electrical characteristics, it is possible to use a potential difference between the electrode 30 and another electrode that is in contact with another site of a living body. The electrode 30 is disposed to be exposed from a distal end 13 of the annular expansive portion 12, and the distal end 13 of the annular expansive portion 12 is pressed against the inner wall of the biological lumen. In this manner, the electrode 30 can be brought into contact with the inner wall of the biological lumen.

As illustrated in FIGS. 3 and 4, a central axis O of the ultrasound tester 20 extends along the extending direction of the catheter 40. The ultrasound element 21 transmits and receives the ultrasound wave along the radial direction to acquire the inner wall surface information around the central axis O. In other words, the ultrasound element 21 acquires information on a periphery of the catheter 40 (hereinafter referred to as “peripheral information”) along a plane orthogonal to the extending direction of the catheter 40. Specifically, the ultrasound element 21 has an ultrasound transmitter and an ultrasound receiver, transmits the ultrasound wave in the radial direction by using the ultrasound transmitter, and receives the reflected ultrasound wave by using the ultrasound receiver. The ultrasound element 21 performs signal processing to acquire a signal relating to information. The ultrasound element 21 can acquire the inner wall surface information by receiving the ultrasound wave reflected from the biological lumen in a state where the ultrasound element 21 is inserted into the biological lumen.

As illustrated in FIG. 4, the shaft 22 fixes the ultrasound element 21 in the distal end along the central axis O. The shaft 22 is rotatable around the central axis O along the circumferential direction of the catheter 40. The ultrasound element 21 rotates around the central axis O in conjunction with the rotation of the shaft 22. In this manner, the ultrasound element 21 can acquire the peripheral information around the central axis O. In addition, the shaft 22 is movable along the central axis O, that is, along the extending direction of the catheter 40. The ultrasound element 21 moves along the central axis O in conjunction with the movement of the shaft 22 along the central axis O. In this manner, the ultrasound element 21 can acquire the peripheral information along the central axis O. When the shaft 22 moves along the central axis O, the tube 23 also moves in conjunction with the movement of the shaft 22. The outer diameter of the shaft 22 is smaller than the outer diameter of the ultrasound element 21.

As illustrated in FIG. 4, the tube 23 is a flexible tubular member that covers the shaft 22 in the circumferential direction. Since the tube 23 is in close contact with the shaft 22, the tube 23 can slide in the extending direction with respect to the catheter 40 without hindering the rotation and movement of the shaft 22. In addition, the proximal portion of the tube 23 is harder than the distal portion of the tube 23 so that a hand-side pushing force on the proximal side of the ultrasound tester 20 is easily transmitted to the distal side of the ultrasound tester 20.

As illustrated in FIGS. 3 and 4, the catheter 40 is a rapid exchange (RX) type catheter which has a distal portion 45 that is an end portion on the distally located side and a proximal portion (not illustrated) that is an end portion on the proximally located side, and in which the second lumen 42 is partitioned only in the distal portion 45. The catheter 40 is not limited to the RX type catheter, and may be a catheter having other shapes, for example, an over-the-wire (OTW) type catheter.

### [Method for Generating Three-Dimensional Image of Heart]

FIG. 5 is a flowchart illustrating details of a method for generating the three-dimensional image of the heart. FIG. 6(a) is a graph illustrating a time change in intensity of an arterial pulse measured by an arterial pulse measurement device. FIG. 6(b) is a graph illustrating a time change in intensity of an electric potential which results from electrical activity of the heart measured by an electrocardiograph. FIG. 7 is a graph obtained by compressing the graph illustrated in FIG. 6(a) or 6(b) along a time axis.

As illustrated in FIG. 5, the image processing device 1 receives an input of information on the inner wall surface (inner wall surface information) of the heart acquired by the ultrasound element 21 as the peripheral information acquisition device at different times, via the information input unit 76 (Step S101). Specifically, the ultrasound element 21 is rotated at a predetermined rotation speed (for example, 1,800 rpm) along the circumferential direction of the catheter 40 by the drive unit 50. While moving at a predetermined speed (for example, 0.5 mm/sec) along the extending direction of the catheter 40, the ultrasound element 21 acquires the inner wall surface information at a plurality of positions on the inner wall surface of the heart at a predetermined time interval (for example, 1/30 seconds). The ultrasound element 21 may move only in one direction along the extending direction of the catheter 40, or may repeatedly reciprocate.

The image processing device 1 receives an input of the pulsation cycle of the heart of the subject which is measured by the pulsation measurement device 160, via the pulsation input unit 60 (Step S102). Specifically, in a case where the pulsation measurement device 160 is configured to include the arterial pulse measurement device, data indicating a time change in intensity of the arterial pulse as illustrated in FIG. 6(a) is acquired. The intensity of the arterial pulse has a waveform that periodically reaches a peak, and when the intensity of the arterial pulse reaches the peak, the heart is in a systole or a diastole to the maximum in response to the pulsation. A pulsation cycle 101 of the heart can be measured, based on periodicity of the time change in the intensity of the arterial pulse. In addition, in a case where the pulsation measurement device 160 is configured to include the electrocardiograph, data indicating a time change in the intensity of the electric potential which results from electrical activity of the heart as illustrated in FIG. 6(b) is acquired. The intensity of the electric potential has a waveform that periodically reaches a peak. For example, when the intensity of the electric potential reaches a peak of an R-wave, an electric signal for bringing the heart into the systole is generated. The pulsation cycle 101 of the heart can be measured, based on periodicity of the time change in the intensity of the electric potential. As illustrated in FIGS. 6(a) and 6(b), the pulsation cycle 101 of the heart is a time for one repetition unit in which the heart is repeatedly in the systole and in the diastole in response to the pulsation. The image processing device 1 may use the control unit 54 to estimate the pulsation cycle of the heart as follows. The image processing device 1 extracts a fluctuation state of the inner wall surface of the heart, for example, such as periodical characteristics appearing in a time history of a position of the inner wall surface, from the plurality of inner wall surface information input at a predetermined time interval in the process of Step S101, and acquires the peak of the systole and the diastole of the heart from the fluctuation state. In this case, the pulsation input unit 60 is configured to fulfill one function of the control unit 54.

Simultaneously with the process of Step S102, the image processing device 1 receives an input of the respiration cycle of the subject which is measured by the respiration measurement device 170, via the respiration input unit 70 (Step S103). For example, the respiration measurement device 170 can be configured to include the electrocardiograph or the arterial pulse measurement device which is common to that of the pulsation measurement device 160. The respiration measurement device 170 may be configured to include a measurement device that can directly measure the respiration cycle of the subject. Specifically, in a case where the respiration measurement device 170 is configured to include the arterial pulse measurement device, a graph illustrated in FIG. 7 is acquired by compressing a graph of a time change in the intensity of the arterial pulse illustrated in FIG. 6(a) along a time axis. In addition, in a case where the respiration measurement device 170 is configured to include the electrocardiograph, a graph similar to the graph illustrated in FIG. 7 is acquired by compressing the R-wave indicating the highest peak in each waveform in the graph of the time change in the intensity of the electric potential illustrated in FIG. 6(b) along the time axis. As illustrated in FIG. 7, the peak of the intensity of the arterial pulse or the peak of the intensity of the electric potential periodically and repeatedly increases and decreases on a long-term basis. It is known that the increase and decrease cycle coincides with a respiration cycle 102 of the subject. Therefore, the respiration cycle 102 of the subject can be measured by measuring the increase and decrease cycle of the peak of the intensity of the arterial pulse or the peak of the intensity of the electric potential. The image processing device 1 can use the control unit 54 to estimate the respiration cycle of the subject as follow. The image processing device 1 extracts a fluctuation state of the inner wall surface of the heart, for example, such as periodical characteristics appearing in a time history of a position of the inner wall surface, from the plurality of inner wall surface information input at a predetermined time interval in the process of Step S101, acquires the peak of the systole and the diastole of the heart from the fluctuation state, and extracts a long-term basis increase and decrease in the peak. In this case, the respiration input unit 70 is configured to fulfill one function of the control unit 54.

The image processing device 1 uses the control unit to select, as selection information, the inner wall surface information input at a timing at which a predetermined pulsation phase range included in the pulsation cycle 101 (refer to FIG. 6) and a predetermined respiration phase range included in the respiration cycle 102 (refer to FIG. 7) overlap each other (Step S104). The predetermined pulsation phase range is a predetermined time range including a predetermined phase in each cycle of the pulsation. In other words, in the predetermined pulsation phase range, a diastole state and a systole state based on the pulsation of the heart are in the same state over each cycle, and are in a state where the influences of the pulsation of the heart on a size, a shape or the like of the heart are close to each other over each cycle. In addition, the predetermined respiration phase range is a predetermined time range including a predetermined phase in each cycle of the respiration. In other words, in the predetermined respiration phase range, the respiration of the subject is in the same state over each cycle, and is in a state where the influences of the respiration on a size, a shape or the like of the heart are close to each other over each cycle. Therefore, the inner wall surface information acquired at the timing at which the predetermined pulsation phase range and the predetermined respiration phase range overlap each other is selected. In this manner, it is possible to acquire the inner wall surface information in which the influences of the pulsation and the respiration on the size, the shape or the like of the heart are uniform. In this example, as illustrated in FIG. 6(a), a predetermined pulsation phase range 103 includes a peak 105 of the pulsation cycle 101. As illustrated in FIG. 7, a predetermined respiration phase range 104 includes a peak 106 of the respiration cycle 102. For example, a peak 107 of a graph illustrated in FIG. 7 indicates a timing at which the peak 105 of the pulsation cycle and the peak 106 of the respiration cycle are simultaneous with each other. That is, as an example of the timing at which the predetermined pulsation phase range included in the pulsation cycle and the predetermined respiration phase range included in the respiration cycle overlap each other, it is possible to adopt a timing of the peak 107 at which the peak 105 of the pulsation cycle and the peak 106 of the respiration cycle are simultaneous with each other. FIG. 15 schematically illustrates the timing of the peak 107 as an example of the timing at which the predetermined pulsation phase range included in the pulsation cycle and the predetermined respiration phase range included in the respiration cycle overlap each other.

A process of Step S104 will be described in detail with reference to FIGS. 8 to 11. FIG. 8 is a view illustrating a distribution of heart position information acquired by using the ultrasound element 21 as the peripheral information acquisition device. In FIG. 8, a horizontal axis Z indicates a moving distance of the ultrasound element 21 along the extending direction of the catheter 40, and a vertical axis X indicates a distance from the ultrasound element 21 to the inner wall surface of the heart, as the inner wall surface information measured by the ultrasound element 21. These are the same in FIGS. 9 to 11. FIG. 9 is a view illustrating a state where the selection information is selected from the inner wall surface information. FIG. 10 is a view illustrating a distribution of the selection information.

As illustrated in FIG. 8, in the process of Step S101, in the image processing device 1, a change in the inner wall surface information input from the ultrasound element 21 via the information input unit 76 greatly fluctuates along the vertical axis X while the horizontal axis Z is slightly changed. The reason is considered as follows. While the ultrasound element 21 moves along the extending direction, the size or the shape of the heart is changed due to pulsation of the heart and the respiration.

In FIG. 8, waveforms corresponding to the peaks of the graph illustrated in FIG. 7 are superimposed and illustrated by a broken line. The horizontal axis Z in FIG. 8 is the moving distance of the ultrasound element 21 along the extending direction of the catheter 40. However, the ultrasound element 21 moves at a constant speed.

Accordingly, the horizontal axis Z can coincide with a time axis which is the horizontal axis in FIG. 7. In FIG. 9, a time of the peak in the lower end of the waveform illustrated by the broken line is illustrated by a straight line along the vertical axis X. The time of the peak in the lower end of the waveform is a timing at which the peak of the pulsation cycle and the peak of the respiration cycle are simultaneous with each other as described above. When the inner wall surface information input at the time of the peak in the lower end of the waveform is selected as the selection information, the inner wall surface information is illustrated as in FIG. 10. As illustrated in FIG. 10, the selection information is selected at the timing at which the peak of the pulsation cycle and the peak of the respiration cycle are simultaneous with each other, the fluctuations in the inner wall surface information along the vertical axis X are reduced.

Instead of the processes of Steps S101 and S104, the image processing device 1 may receive an input of the inner wall surface information, only at a timing at which a first phase of the pulsation cycle and a second phase of the respiration cycle are simultaneous with each other. In other words, the image processing device 1 estimates the timing at which the first phase of the pulsation cycle and the second phase of the respiration cycle are simultaneous with each other, and may receive only the input of the inner wall surface information input from the ultrasound element 21 in accordance with the timing. In this case, the inner wall surface information illustrated in FIG. 10 is directly input to the image processing device 1 from the ultrasound element 21.

The image processing device 1 uses the control unit 54 to perform a complementary process on the inner wall surface information (Step S105). As the complementary process, for example, it is possible to use a machine learning method such as deep learning or a method of complementing missing information from a three-dimensional spline curve (3D spline curve) or discrete data and drawing a smooth curve. FIG. 11 is a view illustrating a distribution in which the complementary process is performed on the discrete data of the inner wall surface information illustrated in FIG. 10.

When the complementary process is performed, the image processing device 1 may select only the inner wall surface information at the timing at which the first phase of the pulsation cycle and the second phase of the respiration cycle are simultaneous with each other, and may construct the two-dimensional image, based on the selected inner wall surface information. Furthermore, the image processing device 1 may acquire a position or an area of the heart or a position or an area of the medical instrument inserted into the heart, from the constructed two-dimensional image. On the other hand, data of the non-selected area is missing data. However, the missing data is interpolated by performing the complementary process on the information on the position and the area of the heart by using the control unit 54 (Step S105). As the complementary process, for example, it is possible to use a machine learning method such as deep learning or a method of complementing missing information from a three-dimensional spline curve (3D spline curve) or discrete data and drawing a smooth curve.

The image processing device 1 uses the control unit 54 to generate the two-dimensional image of the heart, based on the inner wall surface information on which the complementary process is performed, and to generate the three-dimensional image, based on the two-dimensional image (Step S106). In this manner, the image processing device 1 ends the process. The image processing device 1 may first generate the two-dimensional image or the three-dimensional image based on the two-dimensional image, based on the inner wall surface information input in the process of Step S101. Thereafter, the image processing device 1 may generate the two-dimensional image complemented through the processes of Step S104 and Step S105, and may generate the three-dimensional image, based on the complemented two-dimensional image.

As described above, in the inner wall surface information acquired by the ultrasound element 21 as the peripheral information acquisition device and input to the information input unit 76, the image processing device 1 selects, as the selection information, the inner wall surface information input at the timing at which the predetermined pulsation phase range included in the pulsation cycle and the predetermined respiration phase range included in the respiration cycle overlap each other. Therefore, mapping is performed on the heart, based on the selection information. In this manner, it is possible to improve accuracy in performing the mapping on the heart, which considers a time-dependent change in a size or a shape of the heart.

### Modification Example of Method for Generating Three-Dimensional Image of Heart

FIG. 12 is a flowchart illustrating a modification example of a method for generating the three-dimensional image of the heart. As illustrated in FIG. 12, the image processing device 1 acquires the information on the inner wall surface (inner wall surface information) of the heart which is acquired by the ultrasound element 21 as the peripheral information acquisition device, via the information input unit 76 (Step S201).

The image processing device 1 receives an input of the pulsation cycle of the heart of the subject which is measured by the pulsation measurement device 160, via the pulsation input unit 60 (Step S202). Specifically, the image processing device 1 can receive the input of the pulsation cycle of the heart of the subject by using a method the same as that of the process in Step S102 illustrated in FIG. 5.

Simultaneously with the process of Step S202, the image processing device 1 receives the input of the respiration cycle of the subject which is measured by the respiration measurement device 170, via the respiration input unit 70 (Step S203). Specifically, the image processing device 1 can receive the input of the respiration cycle of the subject by using a method the same as that of the process in Step S103 illustrated in FIG. 5.

The image processing device 1 uses the control unit 54 to classify the inner wall surface information input in the process of Step S201 into a group of a combination between the pulsation phase range and the respiration phase range when the inner wall surface information is input (Step S204). For example, the image processing device 1 classifies the inner wall surface information into the group by associating an identification number for each group with the inner wall surface information and storing the information in the storage unit 53. Here, the pulsation phase range is a predetermined time range including a predetermined phase in each cycle of the pulsation, and each cycle of the pulsation includes a plurality of pulsation phase ranges. The above-described predetermined pulsation phase range is one of the plurality of pulsation phase ranges. In addition, the respiration phase range is a predetermined time range including a predetermined phase in each cycle of the respiration, and each cycle of the respiration includes a plurality of respiration phase ranges. The above-described predetermined respiration phase range is one of the plurality of respiration phase ranges.

Details of the pulsation phase range and the respiration phase range will be described with reference to FIGS. 13 and 14. FIG. 13 is a view for describing the pulsation phase range by using the graph indicating the time change in the intensity of the arterial pulse illustrated in FIG. 6(a). FIG. 14 is a view for describing the respiration phase range by using the graph illustrated in FIG. 7. As illustrated in FIG. 13, for example, the pulsation phase range can include a systole T1 including a peak P1 of the systole, a diastole T2 including a peak P2 of the diastole, and an intermediate period T3 after the diastole T2 and before the subsequent systole T1. As illustrated in FIG. 14, for example, the respiration phase range can include an expiration period T4 including a peak P4 of expiration and an inspiration period T5 including a peak P5 of inspiration. In this case, the number of the plurality of pulsation phase ranges in each cycle of the pulsation is three (T1, T2, and T3), and the number of the plurality of respiration phase ranges in each cycle of the respiration is two (P4 and P5). Accordingly, there are six combinations between the pulsation phase range and the respiration phase range. The combination includes at least a maximum diastole in which the heart is in the diastole to the maximum or a minimum systole in which the heart is in the systole to the maximum. For example, the maximum diastole represents a case where the pulsation phase range is the diastole T2 and the respiration phase range is the inspiration period T5. For example, the minimum systole represents a case where the pulsation phase range is the systole T1 and the respiration phase range is the expiration period T4. FIG. 13 illustrates an example in which the plurality of pulsation phase ranges are set at a time interval from each other. However, all time points may be set to be included in any one of the pulsation phase ranges without any time interval. In a case where the plurality of pulsation phase ranges are set at a time interval from each other, and in a case where a time point when the inner wall surface information is input does not correspond to any one of the pulsation phase ranges, the process may return to Step S201. Alternatively, it may be determined that the time point corresponds to the closest pulsation phase range on the time axis. In addition, FIG. 14 illustrates the example in which the plurality of respiration phase ranges are set without the time interval from each other. However, the plurality of respiration phase ranges may be set to have the time interval from each other. In a case where the plurality of respiration phase ranges are set at a time interval from each other, and in a case where a time point when the inner wall surface information is input does not correspond to any one of the respiration phase ranges, the process may return to Step S201. Alternatively, it may be determined that the time point corresponds to the closest respiration phase range on the time axis.

The image processing device 1 uses the control unit 54 to determine whether or not a display mode is set to a time-dependent change following mode (Step S205). Here, the display mode includes the time-dependent change following mode and a fixed mode. For example, any one of the display modes is set in advance, based on the input information received by the operation reception unit 52, and is stored in the storage unit 53. The time-dependent change following mode is a mode in which the image to be displayed on the display unit 51 is frequently switched to the three-dimensional image generated based on the inner wall surface information belonging to the group into which the newly acquired inner wall surface information is classified and the image is displayed. In the time-dependent change following mode, it is possible to monitor a time-dependent change in the size, the shape or the like of the heart. The fixed mode is a mode in which the image to be displayed on the display unit 51 is fixed to the three-dimensional image generated based on the inner wall surface information belonging to a pre-selection group (hereinafter, appropriately referred to as a “selection group”). In the fixed mode, it is possible to always display the heart in a predetermined state, such as the heart which is in the diastole state due to the pulsation, for example. In a case where the image processing device 1 determines that the display mode is set to the time-dependent change following mode (Yes in Step S205), the image processing device 1 proceeds to the process of Step S206.

The image processing device 1 uses the control unit 54 to generate the two-dimensional image, based on the plurality of inner wall surface information belonging to the group into which the newly input inner wall surface information is classified, and generates the three-dimensional image, based on the two-dimensional image (Step S206). Specifically, in a case where the three-dimensional image previously generated based on the inner wall surface information other than the newly input inner wall surface information in the plurality of inner wall surface information belonging to the group is stored in the storage unit 53, the image processing device 1 reads the previously generated three-dimensional image, and generates a new three-dimensional image acquired by adding the newly input inner wall surface information. When generating the three-dimensional image in this step, the image processing device 1 may perform the complementary process as in the process of Step S106 illustrated in FIG. 5.

After the process of Step S206, the image processing device 1 uses the control unit 54 to sequentially switch to and display the newly generated three-dimensional image on the display unit 51 (Step S207). At this time, the image processing device 1 may store the newly generated three-dimensional image in the storage unit 53, as the three-dimensional image of the group into which the newly input inner wall surface information is classified. The image processing device 1 proceeds to the process of Step S211 after the process of Step S207.

On the other hand, in a case where the image processing device 1 determines in the determination process of Step S205 that the display mode is not set to the time-dependent change following mode, that is, the display mode is set to the fixed mode (No in Step S205), the image processing device 1 proceeds to the process of Step S208.

The image processing device 1 uses the control unit to determine whether or not the group into which the newly input inner wall surface information is classified is the selection group (Step S208). Here, the selection group means one group selected from the groups of combination between the pulsation phase range and the respiration phase range. For example, the selection group is set in advance, based on the input information received by the operation reception unit 52, and is stored in the storage unit 53. In a case where the image processing device 1 determines that the group into which the newly input inner wall surface information is classified is the selection group (Yes in Step S208), the image processing device 1 proceeds to the process of Step S209. On the other hand, in a case where the image processing device 1 determines that the group into which the newly input inner wall surface information is classified is not the selection group (No in Step S208), the image processing device 1 proceeds to the process of Step S211.

In the process of Step S209, the image processing device 1 uses the control unit 54 to generate the two-dimensional image, based on the plurality of inner wall surface information belonging to the selection group, and generates the three-dimensional image, based on the two-dimensional image (Step S209). Specifically, in the plurality of inner wall surface information belonging to the selection group, the three-dimensional image previously generated based on the inner wall surface information other than the newly input inner wall surface information is read from the storage unit 53, and the new three-dimensional image acquired by adding the newly input inner wall surface information is generated. When generating the three-dimensional image in this step, the image processing device 1 may perform the complementary process as in the process of Step S106 illustrated in FIG. 5.

After the process of Step S209, the image processing device 1 uses the control unit 54 to update and display the newly generated three-dimensional image on the display unit (Step S210). Specifically, in a case where the display mode is the fixed mode, the display unit 51 displays the three-dimensional image generated based on the plurality of inner wall surface information belonging to the selection group. Accordingly, instead of the displayed three-dimensional image, the image processing device 1 updates and displays the new three-dimensional image generated by adding the newly input inner wall surface information. The image processing device 1 proceeds to the process of Step S211 after the process of Step S210.

In the process of Step S211, the image processing device 1 determines whether or not an end operation is input (Step S211). For example, the end operation is input by the input information received by the operation reception unit 52. In a case where the image processing device 1 determines that the end operation is not input (No in Step S211), the image processing device 1 returns to the process of Step S201. On the other hand, in a case where the image processing device 1 determines that the end operation is input (Yes in Step S211), the image processing device 1 ends the process.

Incidentally, in the process of Step S205 illustrated in FIG. 12, an example has been described in which the time-dependent change following mode or the fixed mode is set based on the display mode set in advance. However, the present invention is not limited to the above-described configuration. For example, in a case where the number of the input inner wall surface information is equal to or smaller than a predetermined number, the fixed mode may be set, and in a case where the number of the input inner wall surface information exceeds the predetermined number, the time-dependent change following mode may be set. In a case where the time-dependent change needs to be monitored, the time-dependent change following mode is more preferable than the fixed mode. However, a load becomes heavier in the process. While the number of the input inner wall surface information is small, there is little information that can be monitored. Therefore, there is little advantage in setting the time-dependent change following mode. However, as the number of the input inner wall surface information increases, the advantage increases in setting the time-dependent change following mode. Therefore, according to the above-described configuration, the load in the process can be reduced, and a balanced process for monitoring of the time-dependent change can be performed.

As described above, the image processing device 1 classifies the inner wall surface information input at the timing at which the pulsation phase range and the respiration phase range overlap each other out of the plurality of inner wall surface information input from the ultrasound element 21 as the peripheral information acquisition device, into the group of the combination between the pulsation phase range and the respiration phase range when the inner wall surface information is input. Therefore, the mapping is performed on the heart, based on the inner wall surface information classified into each group. In this manner, it is possible to improve accuracy in performing the mapping on the heart, which considers the time-dependent change in a size or a shape of the heart.

In addition, as described above, each time the inner wall surface information is input from the peripheral information acquisition device such as the ultrasound tester 20, the image processing device 1 generates the three-dimensional image of the heart, based on the plurality of inner wall surface information belonging to the group into which the inner wall surface information is classified, and sequentially switches and displays the three-dimensional image on the display unit 51. Therefore, the image processing device 1 can display the three-dimensional image generated based on the newest inner wall surface information while changing the three-dimensional image in real time.

In addition, as described above, each time the inner wall surface information belonging to the selection group is input from the peripheral information acquisition device such as the ultrasound tester 20, the image processing device 1 generates the three-dimensional image of the heart, based on the plurality of inner wall surface information belonging to the selection group, and updates and displays the three-dimensional image on the display unit 51. Therefore, the image processing device 1 generates the three-dimensional image, based on only the inner wall surface information belonging to a specific group. Accordingly, the three-dimensional image that is not affected by the movement of the heart can be displayed. In addition, the load in the process can be further reduced compared to when sequentially generating the three-dimensional image, based on the inner wall surface information belonging to each group.

The present invention is not limited to the configurations specified in the above-described respective embodiments, and various modifications can be made within the scope not departing from the gist of the invention disclosed in the appended claims. For example, functions included in each configuration element and each step can be rearranged so that all of these do not logically contradict each other. A plurality of the configuration elements or the steps can be combined into one, or can be divided.

In the above-described embodiment, a configuration has been described in which the ultrasound element 21 of the ultrasound tester 20 is used as the peripheral information acquisition device. However, the invention is not limited to this configuration. For example, an apparatus including an image sensor element as the peripheral information acquisition device may be used instead of the ultrasound tester 20. Examples of the apparatus using the image sensor element include an optical coherence tomography apparatus, an optical frequency domain imaging apparatus for diagnosis, or an endoscope. In a case of using the optical coherence tomography apparatus or the optical frequency domain imaging apparatus for diagnosis, an imaging core unit that emits light toward the inner wall surface of the heart to detect the reflected light can be used as the image sensor element. In a case of using the endoscope, a light-receiving element such as a CCD sensor or a CMOS sensor that receives light from a subject and converts the light into an electric signal corresponding to intensity of the light can be used as the image sensor element.

### INDUSTRIAL APPLICABILITY

The present disclosure relates to an information selection device.

### REFERENCE SIGNS LIST

1: image processing device (information selection device)
2: medical device
10: guide wire
11: linear portion
12: annular expansive portion
13: distal end of annular expansive portion
20: ultrasound tester
21: ultrasound element (peripheral information acquisition device)
22: shaft
23: tube
30, 30a to 30j: electrode
40: catheter (tubular member)
41: first lumen
42: second lumen
45: distal portion
46: opening
50: drive unit
51: display unit
52: operation reception unit
53: storage unit
54: control unit
60: pulsation input unit
70: respiration input unit
76: information input unit
101: pulsation cycle
102: respiration cycle
103: predetermined pulsation phase range
104: predetermined respiration phase range
105: peak of pulsation cycle
106: peak of respiration cycle
160: pulsation measurement device
170: respiration measurement device
A: radial direction of guide wire
B: circumferential direction of guide wire
O: central axis of ultrasound tester
P1: peak of systole
P2: peak of diastole
P4: peak of expiration
P5: peak of inspiration
T1: systole (pulsation phase range)
T2: diastole (pulsation phase range)
T3: Intermediate period (pulsation phase range)
T4: expiration period (respiration phase range)
T5: inspiration period (respiration phase range)

1. An information selection device comprising:
a pulsation input unit capable of inputting a pulsation cycle of a heart of a subject;
a respiration input unit capable of inputting a respiration cycle of the subject;
an information input unit capable of inputting inner wall surface information on the heart at different times; and
a control unit that selects, as selection information, the inner wall surface information input at a timing at which a predetermined pulsation phase range included in the pulsation cycle and a predetermined respiration phase range included in the respiration cycle overlap each other, out of a plurality of the input inner wall surface information.

2. The information selection device according to claim 1,
wherein the predetermined pulsation phase range includes a peak of the pulsation cycle.

3. The information selection device according to claim 1,
wherein the predetermined respiration phase range includes a peak of the respiration cycle.

4. The information selection device according to claim 1,
wherein the control unit generates a three-dimensional image of the heart, based on a plurality of the selection information.

5. The information selection device according to claim 1,
wherein the control unit classifies the inner wall surface information input at a timing at which each of a plurality of pulsation phase ranges having one predetermined pulsation phase range and each of a plurality of respiration phase ranges having one predetermined respiration phase range overlap each other, out of the plurality of inner wall surface information, into a group for each combination.

6. The information selection device according to claim 5,
wherein the control unit generates a three-dimensional image of the heart, based on a plurality of inner wall surface information classified into the same group.

7. The information selection device according to claim 6, further comprising:
a display unit,
wherein each time the inner wall surface information is input to the information input unit, the control unit generates the three-dimensional image of the heart, based on the plurality of inner wall surface information belonging to a group into which the inner wall surface information is classified, and displays the three-dimensional image on the display unit.

8. The information selection device according to claim 6, further comprising:
a display unit,
wherein each time the inner wall surface information belonging to a pre-selection group is input to the information input unit, the control unit generates the three-dimensional image of the heart, based on a plurality of information belonging to the pre-selection group, and displays the three-dimensional image on the display unit.

9. An information selection device comprising:
a pulsation input unit capable of inputting a pulsation cycle of a heart of a subject;
a respiration input unit capable of inputting a respiration cycle of the subject; and
an information input unit capable of inputting inner wall surface information on the heart at different times,
wherein the information input unit receives an input of only the inner wall surface information at a timing at which a predetermined pulsation phase range included in the pulsation cycle and a predetermined respiration phase range included in the respiration cycle overlap each other.

10. The information selection device according to claim 1,
wherein the information input unit receives an input of the inner wall surface information acquired by an ultrasound element or an image sensor element located inside a tubular member inserted into the heart, and
wherein the information selection device further comprises a drive unit that moves the ultrasound element or the image sensor element inside the tubular member.

11. The information selection device according to claim 10,
wherein the information input unit includes the ultrasound element, and
wherein the ultrasound element irradiates an inner wall surface of the heart with an ultrasound wave, and is capable of acquiring the inner wall surface information on the heart, based on the ultrasound wave reflected from the inner wall surface of the heart.

12. The information selection device according to claim 11,
wherein the drive unit is connected with a proximal portion of a shaft,
wherein the ultrasound element is fixed to a distal portion of the shaft, and
wherein the drive unit moves the ultrasound element via the shaft.

13. The information selection device according to claim 9,
wherein the information input unit receives an input of the inner wall surface information acquired by an ultrasound element or an image sensor element located inside a tubular member inserted into the heart, and
wherein the information selection device further comprises a drive unit that moves the ultrasound element or the image sensor element inside the tubular member.

14. The information selection device according to claim 13,
wherein the information input unit includes the ultrasound element, and
wherein the ultrasound element irradiates an inner wall surface of the heart with an ultrasound wave, and is capable of acquiring the inner wall surface information on the heart, based on the ultrasound wave reflected from the inner wall surface of the heart.

15. The information selection device according to claim 14,
wherein the drive unit is connected with a proximal portion of a shaft,
wherein the ultrasound element is fixed to a distal portion of the shaft, and
wherein the drive unit moves the ultrasound element via the shaft.
